(19) United States
(12) Patent Application Publication
(10) Pub. No.: US 2003/0040794 A1
(43) Pub. Date: Feb. 27, 2003

(54) Extra-pericardium heart assist device and method

(76) Inventor: Kevin Jon Lawson, Redding, CA (US)

Correspondence Address: Robert Charles Hill, 235 Montgomery Street # 821, San Francisco, CA, 94104, US

(21) Appl. No.: 09/935,261

(22) Filed: Aug. 22, 2001

(51) Int. Cl. A61M 1/12

(52) U.S. Cl. 623/3.11

(57) ABSTRACT

A heart prosthesis comprises a pulsating mechanism that is surgically placed inside the chest cavity between the sternum and pericardial sac. The mechanism's surfaces expand outward and contract inward to knead the heart in step with its natural rhythms. Electromagnetic repulsion and attraction is used to pulse the mechanism surfaces, and a control circuit is connected to drive an electromagnet.

### BACKGROUND OF THE INVENTION

1. Field of the Invention

The present invention relates to surgical methods and devices that mechanically assist human heart pumping, and more specifically those that can be permanently placed inside the chest cavity but outside the pericardium.

2. Description of Related Art

Heart failure is a severe condition that can render the heart incapable of pumping an adequate supply of blood to the body. The chambers and valves are generally well functional, but often get discarded with current procedures like artificial heart devices or organ-donor transplants.

The heart works within the chest cavity and is supported by the diaphragm and ligaments. When the heart contracts, the cavities are conical or cone shaped, the diameter of the cone is decreased and the height is decreased. The heart should contract completely and eject a maximum volume of blood and then quickly refill.

One of the problems in heart failure patients is that even though their hearts beat faster, each beat only moves a small volume of blood. So it falls behind on the body's needs. One of the compensatory mechanisms of the heart that is having trouble pumping the required volume of blood is that when the percentage of ejection is going down it tends to dilate so that the entire heart volume gets enlarged. Sick hearts tend to be big hearts, or enlarged hearts. It is not because the muscles are really getting bigger, it is because the way the heart is made the output of blood has to stay at a reasonable stable level. So the total volume of the heart increases even though the percentage of blood ejection becomes smaller. But when the heart compensates by enlarging too much it is not good for the heart long-term. Hearts that dilate or enlarge tend to gradually become weaker and fail more.

Conventional medical treatments for congestive failure or heart failure usually begin with medications. People who do not respond to medications are graduated to cardiac transplants, artificial heart devices, and ventricular assist devices. The latter are usually interim to transplants. A blood pump is implanted into the body and helps move blood from the heart to the rest of the body. Balloon pumps also have been used which use a catheter passed in through a blood vessel in the groin and threaded up near the heart. A balloon is passed inside the blood vessel and is sequentially inflated and deflated to assist the heart in pumping blood.

Current treatments introduce artificial materials into the blood stream. Any materials in contact with the blood tend to cause blood clotting. Unfortunately, any blood clots that form here can break off and go downstream to cause major problems in the lungs and brain, e.g., stroke and pulmonary embolism. The prior art responds to this by placing the patients on blood thinners or anti-coagulation medicine. But this subjects patients to chronic bleeding, ulcers, strokes, etc. Conventional artificial hearts and heart assist devices also need relatively large amounts of operating power, so patients are often tethered to a power-pack or other power source.

### SUMMARY OF THE INVENTION

An object of the present invention is to provide a prosthetic device that improves and supports adequate blood flow from the heart without surgical invasion of the heart and its pericardial sac.

Another object of the present invention is to provide a prosthesis for the heart that can be relied upon for permanent use.

Another object of the present invention is to provide a technique for secure anchoring of a cardiac assist device to the skeletal elements of the thoracic cavity to include the sternum, ribs and thoracic vertebra as needed.

Briefly, a prosthesis embodiment of the present invention comprises a pulsating mechanism that is surgically placed inside the chest cavity between the sternum and pericardial sac. The mechanism's surfaces expand outward and contract inward to knead the heart in step with its natural rhythms. Electromagnetic repulsion and attraction is used to pulse the mechanism surfaces, and a control circuit is connected to drive an electromagnet.

An advantage of the present invention is that a prosthetic heart assistant is provided that consumes only that operating power which is necessary to make up deficits in the otherwise unassisted blood flow of the heart.

Another advantage of the present invention is that a prosthesis is provided that can be permanently emplaced within the chest cavity.

A further advantage of the present invention is a heart prosthesis is provided that avoids surgical invasion of the heart itself and thus promotes quicker patient recovery with fewer serious post-operative complications. The positioning of the device outside the pericardium makes abrasion of the heart muscle and sensitive tissues much less likely. The dense pericardium naturally protects the heart and is preserved as a protective sleeve between the heart and the device.

The above and still further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIG. 1 is a cross sectional diagram of a heart patient representing the placement of a single heart prosthesis embodiment of the present invention;

FIG. 2 is a cross sectional diagram of a heart patient representing the alternative placement of two heart prostheses in an embodiment of the present invention;

FIG. 3 is a schematic diagram of a heart prosthesis embodiment of the present invention that is powered through the skin with infrared light and a photovoltaic cell;

FIG. 4 is a schematic diagram of a heart prosthesis embodiment of the present invention that is powered through the skin with magnetic induction;

FIG. 5 is a schematic diagram of a heart prosthesis embodiment of the present invention that is powered through the skin with a battery; and

FIG. 6 is a schematic diagram of a heart prosthesis embodiment of the present invention that is powered through the skin with a battery and uses an electromagnet combined with a permanent magnet.

### DETAILED DESCRIPTION OF THE INVENTION

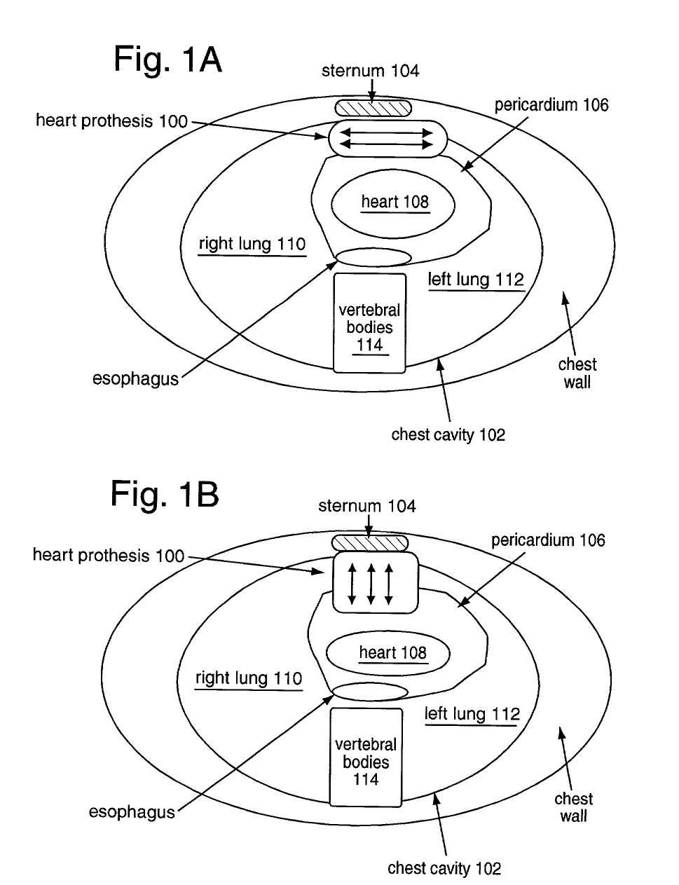

FIGS. 1A and 1B illustrate a typical placement of a heart prosthesis embodiment of the present invention, referred to herein by the reference numeral 100. The heart prosthesis 100 is surgically placed inside a human chest cavity 102 between a sternum 104 and a fibrous pericardium 106. It preferably has a flexible housing, e.g., made of GORTEX or similar bio-compatible and flexible material. During surgical implant, a patient's failed or failing heart 108 is left undisturbed inside the pericardium 106. The other surrounding structures of the body in FIG. 1A and 1B include a right lung 110, a left lung 112, and vertebral bodies (spine) 114.

In operation, heart prosthesis 100 periodically contracts front-to-back as in FIG. 1A, and expands front-to-back as in FIG. 1B, as indicated by arrows. The volume of heart prosthesis 100 remains the same, only its shape changes with regular pulsations. The heart 108 is able to shrink in volume as it expels its charge of blood. The pulsations of heart prosthesis 100 knead the heart 108 and help it both to pump out a full blood volume and draw in a new blood volume.

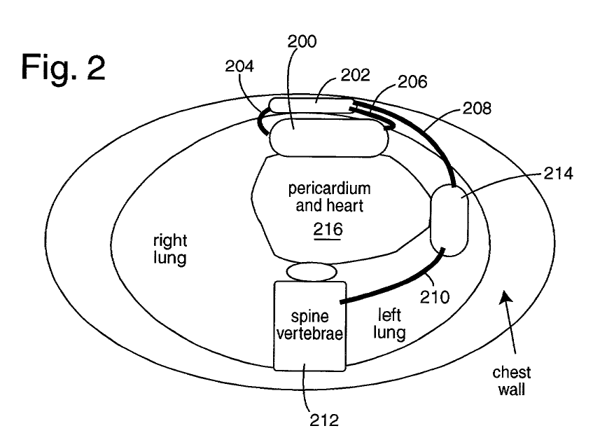

In an alternative embodiment of the present invention, shown in FIG. 2, a heart prosthesis 200 is anchored to a sternum 202 with cables 204 and 206. The use of an anchoring cable 208 to the sternum, and an anchoring cable 210 to the spine 212 would further allow placement of a second heart prosthesis 214 to one side of a pericardium 216. In still further alternative embodiments of the present invention, multiple ones of heart prosthesis 200 and 214 are arrayed around the pericardium 216 and electronically synchronized.

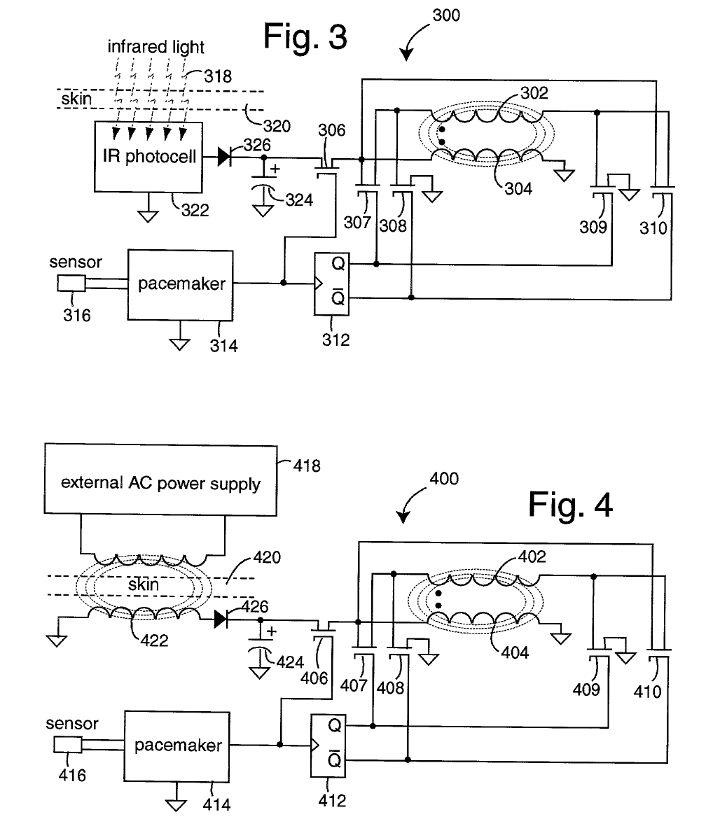

FIGS. 3-6 illustrate a variety of ways that the electronics of heart prosthesis embodiments of the present invention can be constructed. The basic idea amongst all of them is electromagnets and/or permanent magnet combinations are mounted on the internal walls of the prosthesis so their opposing electromotive forces can cause the walls to pulse in and out. At least one electromagnet is energized with direct current (DC) to push its wall out, and then the DC is reversed to pull the wall in. A pacemaker is used to time these actions appropriately. FIGS. 3-6 show three different ways external operating power can be introduced to the heart prosthesis while in place within the chest cavity of a patient.

Referring now to FIG. 3, a heart prosthesis 300 is based on two electromagnets 302 and 304 placed in magnetic opposition to one another. Each are mounted internally on opposite walls of an enclosure to mechanically pull those walls together and force them apart according to the switching-state of several transistors 306-310. Transistor 306 will turn-on electromagnet 304 and supply power to transistors 307 and 310. Electromagnet 302 will turn on with one polarity or the other if transistors 307 and 309 are on, or otherwise if transistors 308 and 310 are on. A flip-flop 312 clocks one pull-in cycle followed by one push-out cycle each time a trigger pulse is received from a pacemaker 314. A surgically placed sensor 316 detects when the heart has been signaled to beat. The heart's own internal structure regularly triggers beats. By the time the heart responds, the electromagnets 302 and 304 come on to assist in the pumping action of the blood. The heart prosthesis 300 is powered by infrared light at wavelengths that pass easily through human tissue and skin 320. A photovoltaic cell 322 converts this to DC electrical power that charges a large storage capacitor 324 through a blocking diode 326. In some embodiments of the present invention, the energy density of capacitor 324 can be increased by using it to store high voltages, e.g., 1 KV and higher.

Referring now to FIG. 4, a heart prosthesis 400 is based on two electromagnets 402 and 404 placed in magnetic opposition to one another. Each are mounted internally on opposite walls of an enclosure to mechanically pull those walls together and force them apart according to the switching-state of several transistors 406-410. Transistor 406 will turn-on electromagnet 404 and supply power to transistors 407 and 410. Electromagnet 402 will turn on with one polarity or the other if transistors 407 and 409 are on, or otherwise if transistors 408 and 410 are on. A flip-flop 412 clocks one pull-in cycle followed by one push-out cycle each time a trigger pulse is received from a pacemaker 414. A surgically placed sensor 416 detects when the atrium has signaled the heart to beat. By the time the ventricle responds, the electromagnets 402 and 404 come on to assist in the pumping action of the blood. The heart prosthesis 400 is powered by an alternating current (AC) power source using magnetic induction that passes easily through human tissue and skin 420. Such induced power is at a frequency and power level that does not interfere with electromagnets 402 and 404. A pickup coil 422 converts this to DC electrical power that charges a large storage capacitor 424 through a blocking diode 426. The pickup coil 422 can be placed well away from the electromagnets 402 and 404. In some embodiments of the present invention, the energy density of capacitor 424 can be increased by using it to store high voltages, e.g., 1 KV and higher.

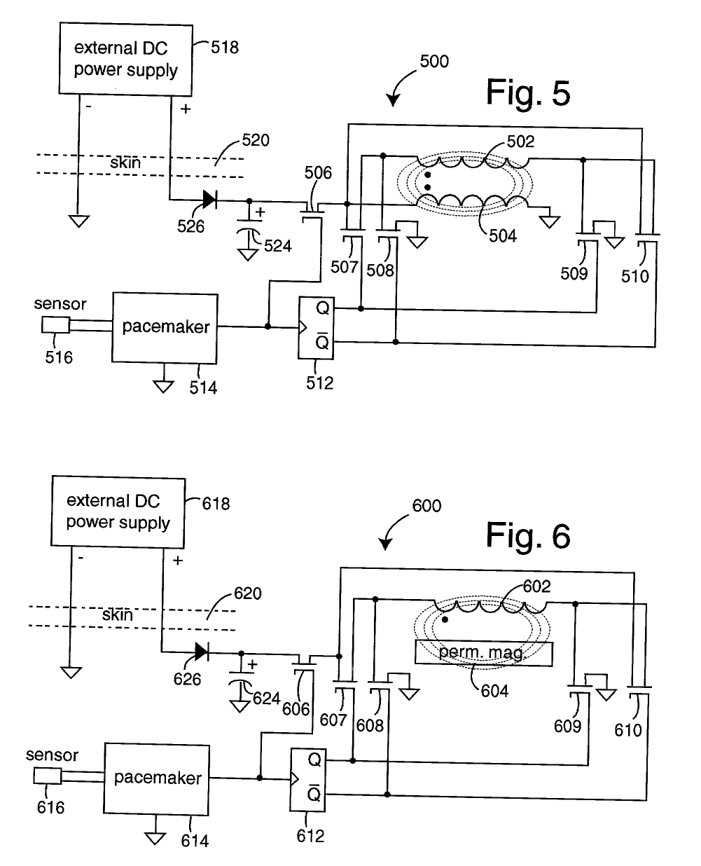

Referring to FIG. 5, a heart prosthesis 500 is based on two electromagnets 502 and 504 placed in magnetic opposition to one another. Each are mounted internally on opposite walls of an enclosure to mechanically pull those walls together and force them apart according to the switching state of several transistors 506-510. Transistor 506 will turn-on electromagnet 504 and supply power to transistors 507 and 510. Electromagnet 502 will turn on with one polarity or the other if transistors 507 and 509 are on, or otherwise if transistors 508 and 510 are on. A flip-flop 512 clocks one pull-in cycle followed by one push-out cycle each time a trigger pulse is received from a pacemaker 514. A surgically placed sensor 516 detects when the brain has signaled the heart to beat. By the time the heart responds, the electromagnets 502 and 504 come on to assist in the pumping action of the blood. The heart prosthesis 500 is powered by a battery 518 with connections that are passed through skin 520. The DC battery power charges a large storage capacitor 524 through a blocking diode 526.

Referring now to FIG. 6, a heart prosthesis 600 is based on one electromagnets 602 and a permanent magnet 604 placed in magnetic opposition to one another. Each are mounted internally on opposite walls of an enclosure to mechanically pull those walls together and force them apart according to the switching-state of several transistors 606-610. Transistor 606 will supply power to transistors 607 and 610. Electromagnet 602 will turn-on with one polarity or the other if transistors 607 and 609 are on, or otherwise if transistors 608 and 610 are on. A flip-flop 612 clocks one pull-in cycle followed by one push-out cycle each time a trigger pulse is received from a pacemaker 614. A surgically placed sensor 616 detects when the brain has signaled the heart to beat. These sensors and pacemakers in general are conventional and universally implanted in heart patients throughout the world.

By the time the heart responds to the signal from the atrium, the electromagnets 602 and 604 come on to assist in the pumping action of the blood. The heart prosthesis 600 is powered by a battery 618 with connections that are passed through skin 620. The DC battery power charges a large storage capacitor 624 through a blocking diode 626.

Embodiments of the present invention are surgically implanted to help patients with severe heart failure or congestive heart failure. It provides assistance to the heart similar to conventional ventricular assist devices. However, embodiments of the present invention are all extracardial and extra-vascular. The native heart tissue is preferably not disturbed, and fits within the chest cavity outside of the pericardium.

Such extracardiac assist device can be a single unit just behind the sternum working with the heart within the chest cavity, or a second unit can be added to the left supported by a sling or supported against a rib to give more direct assistance to the left ventricle of the heart. Two such devices working in concert are also feasible, e.g., retrosternal and left-sided.

Embodiments of the present invention have the benefit of no coagulation problem because they are not in direct contact with the blood flow. It is acting on the patient's own heart to assist it in contracting and expanding by rapidly deploying and then actively retracting. Less power is required. Even in severe heart failures, the heart stills carry much of its work, but it just is not meeting the needs of the body's total requirement. So the power requirement is such that it only assists the native heart. It does not entirely replace the native heart. Possibly an additional thirty percent may be enough of an assist to the native heart to avoid severe symptoms, yet reducing the energy needs of the device.

If heart prosthesis 100 should fail, for example, the patient will not be immediately subjected to a catastrophic failure. But simply slip into heart failure again which can be treated. In contrast, failure of an artificial heart or a ventricular assist device leads to death of the patient.

Measures that improve heart function, even temporarily, can stimulate the heart to recover and improve. Giving the heart muscle a rest leads to a potential for remodeling or reconditioning of the heart.

Embodiments of the present invention are relatively easy to insert or implant because the pericardium is not opened and the surgeon is not directly working on the heart. If the heart has had previous surgery, the doctor does not have to deal with any scars on the heart itself or the major blood vessels. There is no need for bypass devices, or for the heart to be stopped.

Embodiments of the present invention can be implanted through a sternonomy incision in the chest, or by an incision just below the ribs. The right ventricle of the heart is anterior which does less work with a lower pressure system and pumps blood from the body to the lung. The left ventricle is posterior and to the left side. It pumps blood from the lungs to the rest of the body with a high pressure. The left ventricle does most of the work and provides the main blood pressure, both right and left sides must have equal volume output to avoid congestion in the lungs.

The big problem in failure is usually the left ventricle. Embodiments of the present invention push against the sternum and work against the pericardium of the heart, the outer covering of the heart. It resembles cardiopulmonary resuscitation which relies on sternal compression of the heart.

The present invention can stop an enlarged heart from getting even bigger because it helps the heart pump blood. It is even possible that an enlarged heart may return to more normal size.

It is important to remember that the device acts on the pericardium, and not directly on the heart. The pericardium is the fibrous covering that surrounds the heart, and is not particularly sensitive. It is fairly tough, and so it can tolerate the mechanical pressure of the device. Normally the pericardium protects the heart from trauma. The inside of the pericardium is very smooth and holds some fluid which lubricates the heart.

Although particular embodiments of the present invention have been described and illustrated, such was not intended to limit the invention. Modifications and changes will no doubt become apparent to those skilled in the art, and it was intended that the invention only be limited by the scope of the appended claims.

The invention claimed was:

1. A heart prosthesis, comprising:
a flexible housing for placement inside a heart patient's chest cavity next to the fibrous pericardium;
a first electromagnet disposed inside the flexible housing for pushing and pulling a wall in and out in an action that will help the heart pump blood and fill back; and
a control circuit connected to synchronize the movements of the electromagnet with natural heart activity.

2. The heart prosthesis of claim 1, further comprising:
a second electromagnet placed in opposition to the first electromagnet and mounted to an opposite internal wall of the flexible housing.

3. The heart prosthesis of claim 1, further comprising:
a permanent magnet placed in opposition to the first electromagnet and mounted to an opposite internal wall of the flexible housing.

4. The heart prosthesis of claim 1, further comprising:
a power supply connected to feed operating power to the first electromagnet.

5. The heart prosthesis of claim 4, wherein:
the power supply is wirelessly connected to feed operating power to the first electromagnet and does not penetrate the skin with a feed cable.

6. A method for improving heart function in a patient, comprising the steps of:
surgically placing a pulsator mechanism inside a patient's chest cavity between the sternum and the fibrous pericardium;
electromagnetically expanding and contracting said pulsator mechanism to mechanically assist said patient's heart in both expelling blood flow and filling back up again; and
pacing said pulsator mechanism to follow a natural heart beat.

7. The method of claim 6, wherein:
the step of surgically placing results in said pulsator mechanism being positioned between the sternum and the fibrous pericardium.

8. The method of claim 6, wherein:
the step of surgically placing results in said pulsator mechanism being positioned lateral to the fibrous pericardium and anchored to at least one of the sternum and a spine vertebrae.

9. The method of claim 6, wherein:
the step of electromagnetically expanding and contracting depends on electronic switching of at least one electromagnet disposed within the pulsator mechanism.

10. The method of claim 6, wherein:
automatically reverting to natural heart activity in the event of an operational failure of said pulsator mechanism;
wherein, said pulsator mechanism assists but does not prevent continued residual heart activity.
